(19) United States
(12) Patent Application Publication
(10) Pub. No.: US 2011/0217724 A1
(43) Pub. Date: Sep. 8, 2011

(54) METHOD OF PROTECTING CELLS

(75) Inventors: Taina Jaatinen, Helsinki (FI); Seppo Meri, Helsinki (FI); Sami Junnikkala, Helsinki (FI); Jarmo Laine, Helsinki (FI); Jukka Partanen, Helsinki (FI)

(73) Assignee: SUOMEN PUNAINEN RISTI VERIPALVELU, Helsinki (FI)

(21) Appl. No.: 13/123,818

(22) PCT Filed: Oct. 15, 2009
(86) PCT No.: PCT/FI09/50833

§ 371 Date: Apr. 12, 2011

(30) Foreign Application Priority Data
Oct. 15, 2008 (FI) 20085973

(51) Int. Cl. C12Q 1/02; C12N 5/02

(52) U.S. Cl. 435/29; 435/374; 435/347

(57) ABSTRACT

Provided is a method for protecting stem cells in a clinical graft against destruction induced by the complement system by adding to the graft at least one factor capable of inhibiting the complement. Also provided is a method for protecting stem cells in a clinical graft against destruction induced by the complement system using a factor capable of inhibiting the complement.

### FIELD OF THE INVENTION

The present invention relates to a method of protecting stem cells in a clinical graft against the destruction induced by the complement system by adding to the graft at least one factor capable of inhibiting the complement.

The present invention relates also to the use of a factor capable of inhibiting the complement to protect stem cells in a clinical graft against the destruction induced by the complement system. In addition, the present invention relates to a composition or a mixture comprising stem cells and at least one factor capable of inhibiting the complement.

### BACKGROUND OF THE INVENTION

Hematopoietic stem cell (HSC) transplantation is used for treating certain hematological and nonhematological malignant and nonmalignant diseases. Bone marrow and cord blood have been studied, and also used in treating human patients, as stem cell sources. Unfortunately, utilization and success of HSC transplantation suffer from several obstacles such as graft-versus-host disease and graft rejection.

A limiting factor, especially with regard to cord blood transplantation, is the dose of the nucleated cells in the graft. Several approaches to increase the dose of nucleated cells in a graft have been studied including ex vivo expansion of the cells. Also multiunit transplantation and cord blood transplantation supported with infusion of mesenchymal stem cells have been explored in improving the outcome of the transplantation (Grewal, S. S. et al., Blood, 1 Jun. 2003, Vol. 101, No. 11, pp. 4233-4244). In addition, it is known that cord blood cells, such as CD34 negative cells, that are not stem cells are essential for successful engraftment.

In addition, in order to survive in the human body cells must resist the innate and to a large extent also the adaptive immune responses. The cells need to have mechanisms to cope with the complement system, an innate defence mechanism with an ability to opsonize target cells for phagocytosis or kill them directly with the membrane attack complex (MAC). The complement system can be activated, for example, by antibody—antigen complexes or certain foreign structures. For example, nonhuman sialic acid, N-glycolylneuraminic acid (Neu5Gc), incorporated onto a stem cell leads to an immune response mediated by antibodies to Neu5Gc-structure present in most humans.

Sialic acids are a family of acidic saccharides displayed on the surfaces of all cell types, and on several secreted proteins. N-acetylneuraminic acid (Neu5Ac) and N-glycolylneuraminic acid (Neu5Gc) are the two most common mammalian sialic acids. Humans are unable to produce Neu5Gc from NeuAc, which is its metabolic precursor. Human cells are, however, able to take Neu5Gc up from media containing animal derived material and thus also Neu5Gc. Most healthy humans have circulating antibodies specific for Neu5Gc.

In general, human cells are protected against the attack of the complement system by regulator molecules on cell membranes. They include C3b receptor (CR1; CD35), decay accelarating factor (DAF; CD55), membrane cofactor protein (MCP; CD64) and protectin (CD59). In addition, there are soluble proteins in plasma that prevent excessive complement activation in the fluid phase. These include C1 inhibitor (C1INH), factor H (FH), C4b-binding protein (C4 bp), vitronectin (S-protein) and clusterin (SP40,40; apo J) (Springer Semin Immunopathol 15: 369-396 (1994)).

It has now been discovered that stem cells and/or cord blood derived cells are protected against the destruction induced by the complement system with the use of at least one factor capable of inhibiting the complement.

Further, it has now been discovered that stem cells in a clinical graft are protected against the destruction induced by the complement system of the recipient by adding to the graft at least one factor capable of inhibiting the complement.

### BRIEF DESCRIPTION OF THE INVENTION

The present invention relates to a method of protecting cells against the destruction of the complement system with the use at least one factor capable of inhibiting the complement. Specifically, the present invention relates to a method of protecting stem cells and cord blood derived cells against the destruction of the complement system with the use at least one factor capable of inhibiting the complement.

Thus, an object of the present invention is to provide a method of protecting stem cells and cord blood derived cells against the destruction of the complement system with the use at least one factor capable of inhibiting the complement. Another object of the present invention is to provide a method of protecting stem cells in a clinical graft against the destruction induced by the complement system of the recipient by adding to the graft at least one factor capable of inhibiting the complement. Another object of the present invention relates to the use of a factor capable of inhibiting the complement to protect stem cells in a clinical graft against the destruction induced by the complement system. A further object of the present invention relates to a composition or a mixture comprising stem cells and at least one factor capable of inhibiting the complement. Still a further object of the present invention is to provide a method of protecting stem cells against the destruction induced by the complement system, wherein the complement system is activated by a nonhuman Neu5Gc structure on the cell surface, with the use at least one factor capable of inhibiting the complement.

The objects of the invention are achieved by methods, a use and a composition that are characterized by what is stated in the independent claims. The preferred embodiments of the invention are disclosed in the dependent claims.

### BRIEF DESCRIPTION OF THE DRAWINGS

FIG. 1 shows the results of Example 1.

FIG. 2 shows the results of Example 2.

FIG. 3 shows the results of Example 3.

FIG. 4 shows the results of Example 6.

FIG. 5 shows the results of Example 7.

FIG. 6 shows the results of Example 9.

### DETAILED DESCRIPTION OF THE INVENTION

Hematopoietic stem cell (HSC) transplantation is a workable treatment especially for hematological malignant diseases, such as leukaemias. It is used also for the treatment of some hematological nonmalignant and non-hematological malignant and nonmalignant diseases. The success of trans-plantation depends on several matters, one of them being the number of cells in the graft.

Blood from the placenta and/or umbilical cord (referred to cord blood in the present invention) is a rich source for hematopoietic stem cells. A limiting factor with regard to cord blood transplantation is the small size and/or volume of the graft, i.e., the small number of the nucleated cells in the graft. Due to this obstacle cord blood transplantation has been mainly used to treat children, especially small children.

To be successful or optimal, it is necessary that the graft for HSC transplantation contains a sufficient dose of cells relative to recipient size. A dose of 1×10⁶ nucleated cells/kg of the weight of the recipient is currently recommended.

The immune system has a central role in the success of transplantation, especially when human leukocyte antigen-identical sibling donors are not available. The immune system of the host may recognize transplanted cells as foreign, resulting in the rejection of the therapeutic cells. The immunological recognition of the host cells as foreign by the immune cells in the graft is a central obstacle in stem cell transplantation. This results in graft-versus-host disease. The destruction of transplanted cells is primarily thought to be caused by the cellular immunity. However, as demonstrated by the present invention, the cells in the graft can be destroyed by the complement system as well. Hematopoietic stem cells, for example, carry surface structures that are considered to predispose them to immune attack through recognition and direct activation of the complement system.

In one embodiment, the invention is directed to a method for inhibiting the complement-mediated cell killing that results from recipient's antibodies that are recognizing, and binding to, the Neu5Gc glycostructure on stem cells of the graft. It is known in the literature that human stem cells selectively acquire the non-human Neu5Gc structure from e.g. cell culture or ingested food. Also, it is known that many individuals have developed antibodies against the structure. Hence, in stem cell transplantation these antibodies can bind onto the Neu5Gc structures on stem cells of the graft and like other antibodies bound to their targets, they can activate the complement system. Accordingly, a major part of the cells of a graft are devastated by the actions of the immune system before they are transferred to their actual location in the body and have started to grow.

Hematopoietic stem cells (HSC) having ability to form multiple cell types and ability to self-renew, are currently used for treating certain hematological and nonhematological diseases. HSCs can be derived for example from bone marrow and cord blood. Mesenchymal stem cells (MSC) have the potential to differentiate into various cellular lineages and can be expanded in culture conditions without losing their multipotency. Therefore, they present a valuable source for applications in cell therapy and tissue engineering. MSCs can be derived for example from bone marrow.

In addition to hematopoietic and mesenchymal stem cells, the present invention can be used in therapies with other stem cells. Examples of such cells are, in particular, induced pluripotent stem (iPS) cells. iPS cells are a type of pluripotent stem cell derived or produced from principally any adult non-pluripotent or differentiated cell type, such as an adult somatic cell, that has been induced to have all essential features of embryonic stem cells (ESC). The techniques were first described in human cells by Takahashi et al. in Cell 131: 861-872, 2007. Their therapeutic potential has been predicted to be enormous because patients own cells can be induced and hence, ethical and histocompatibility problems can be avoided.

Other cell types to which the present invention aims, include, but are not limited to, embryonal stem cells and/or epithelial stem cells. In technologies for harvesting hESCs the embryo is either destroyed or not, i.e. it remains alive. In one embodiment of the invention, the hESCs are harvested by a method that does not include the destruction of a human embryo.

It has now been observed that mesenchymal stem cells and cord blood-derived mononuclear cells, including the CD34-positive hematopoietic stem cells and CD34-negative more mature cells, are sensitive to complement-mediated destruction. This complement-sensitivity may be due to the scarcity of many key complement inhibitors, such as factor H (FH), complement receptor 1 (CR1, CD35), membrane cofactor protein (MCP, CD46) and decay accelerating factor (DAF) on the surface of these cells. Now, it has been discovered that the complement-mediated cell destruction can be significantly diminished by complement inhibitors, i.e., factors capable of inhibiting the complement, such as FH, CR1, MCP and DAF.

Thus, in one embodiment of the present invention, a method of protecting a stem cell and/or a cord blood derived cell against the destruction of the complement system with the use of at least one factor capable of inhibiting the complement, is provided. In another embodiment of the present invention, a method of protecting a stem cell and/or a cord blood derived cell against the destruction of the complement system, wherein the complement system is activated by a nonhuman Neu5Gc structure on the cell surface, with the use of at least one factor capable of inhibiting the complement, is provided.

In a further embodiment of the present invention, a method of protecting stem cells in a clinical graft against destruction induced by complement system by adding to the graft at least one factor capable of inhibiting the complement, is provided. In still one embodiment of the present invention a method of protecting stem cells in a clinical graft against destruction induced by complement system, wherein the complement system is activated by a nonhuman Neu5Gc structure on the cell surface, by adding to the graft at least one factor capable of inhibiting the complement, is provided.

In one embodiment of the invention, the method of protecting cells against the destruction of the complement system with the use of at least one factor capable of inhibiting the complement is in vitro method. In another embodiment of the invention, the method of protecting cells against the destruction of the complement system with the use of at least one factor capable of inhibiting the complement is in vivo method.

Further, the present invention relates to a composition or a mixture comprising stem cells and at least one factor capable of inhibiting the complement. In one embodiment of the invention, the factor capable of inhibiting the complement in said composition or mixture is selected from factor H, CR1, MCP and DAF. In another embodiment of the invention, the stem cells in said composition or mixture are selected from mesenchymal stem cells, hematopoietic stem cells and/or iRS cells.

The effective amount or dose of the complement inhibitor depends on the inhibitor itself and on the cells in question, for example. In one embodiment of the invention, the inhibitor is used in a concentration range of 50-1000 μg/ml, specifically in a concentration range of 100-750 μg/ml. In another embodiment of the invention factor H is used in a concentration range of 50-1000 μg/ml, specifically in a concentration range of 100-750 μg/ml. Another way of expressing the effective amount or dose of a complement inhibitor is to determine the quantity of the inhibitor per the number of cells in the graft.

Thus, the present invention provides a new way for protecting stem cells, especially mesenchymal and hematopoietic stem cells, and cord blood derived mononuclear cells against the destruction induced by the complement system. The present invention also discloses a way to improve the outcome of stem cell transplantation, in particular, enhanced engraftment. Furthermore, it provides means to use a smaller cell number or graft in the transplantation.

The present invention can be utilized in enabling the use of cord blood transplantation for adult patients and/or patients having weight more than the currently accepted critical dose of nucleated cells in the graft per the weight of the recipient allows.

Cord blood preparation or graft may contain in addition to stem cells all types of blood cells in the cord blood plasma. It is typical and characteristic to cord blood that it comprises nucleated red blood cells and hematopoietic stem cells that are lacking from adult peripheral blood. When prepared 20% HES (hydroxyethylstarch) and 20% DMSO (dimethyl sulfoxide) are normally added to the preparation or graft. Cord blood is collected into a bag containing typically also CPD (citrate phosphate dextrose)-anticoagulant. A cord blood unit may be stored in freezer or liquid nitrogen. Similarly, a graft derived from bone marrow contains also a mixture of other cells in addition to hematopoietic stem cells. The entire mixture of cells can be used as a clinical graft without further processing, alternatively, it may be processed e.g. by removing potentially harmful T-lymphocytes. It is of note that the exact contents of the grafts vary between clinics treating patients.

In addition, the present invention can be utilized in enabling the use of smaller grafts that, for one, contain less potentially harmful T-lymphocytes, that incur and/or are responsible of the graft-versus-host rejection, than grafts having the volume that is calculated based on the dose of nucleated cells in the graft per the weight of the recipient.

It has now been observed that there is individual variation in the complement inhibitor levels, such as factor H level, between different grafts, such as cord blood units. Thus, some cord blood-derived stem cell units may be more prone to complement-mediated lysis than others, for example. This complement sensitivity, based on certain complement inhibitor level in a graft, such as a cord blood unit, could be measured prior to transplantation. Thus, the present invention can be utilized in tailoring the size of the graft to the specific needs, prerequisites and/or requirements of each recipient.

The present invention relates further to a method for determining the need and/or adjusting the amount of fortification of the complement inhibitor by first measuring the concentration and/or amount of said complement inhibitor in the graft and then adding the missing amount of said complement inhibitor thereto or administering it to the recipient separately.

It will be obvious to a person skilled in the art that, as the technology advances, the inventive concept can be implemented in various ways. The invention and its embodiments are not limited to the examples described above but may vary within the scope of the claims.

The following examples illustrate the present invention. The examples are not to be construed to limit the claims in any manner whatsoever.

### Example 1

### Materials and Methods

Cells: Ficoll-Hypaque density gradient was used to isolate mononuclear cells from peripheral blood and cord blood. Bone marrow-derived mesenchymal stem cells were cultured in Minimum Essential Alpha-Medium, supplemented with 20 mM HEPES, 10% FCS, 1× penicillin-streptomycin and 2 mM L-glutamine and plated at the density of 2000-3000/cm². The cells were subcultured until they were fully confluent.

Lysis assay: Labeling of cells was performed by mixing 2×10⁶ cells and 100 μCi of ⁵¹Cr in 1 ml RPMI for 2 h at 37° C. The cells were then washed twice with RPMI, incubated for a further 30 minutes to remove loosely bound ⁵¹Cr and washed twice. Duplicate aliquots of ⁵¹Cr-labeled cells (10⁵ cells/50 μl) were treated with monoclonal antibody against CD59 (YTH53.1) for 20 minutes at 22° C. and with normal human serum (NHS) for 30 minutes at 37° C. in a total volume of 200 μl. NHS was diluted 1:4 and YTH53.1 was used in concentrations 8-67 μg/ml. After centrifugation at 525×g for 5 minutes, 50% of the supernatant was carefully removed and counted in a gamma counter. Cell lysis was determined as percentage of specific release of ⁵¹Cr.

### Results

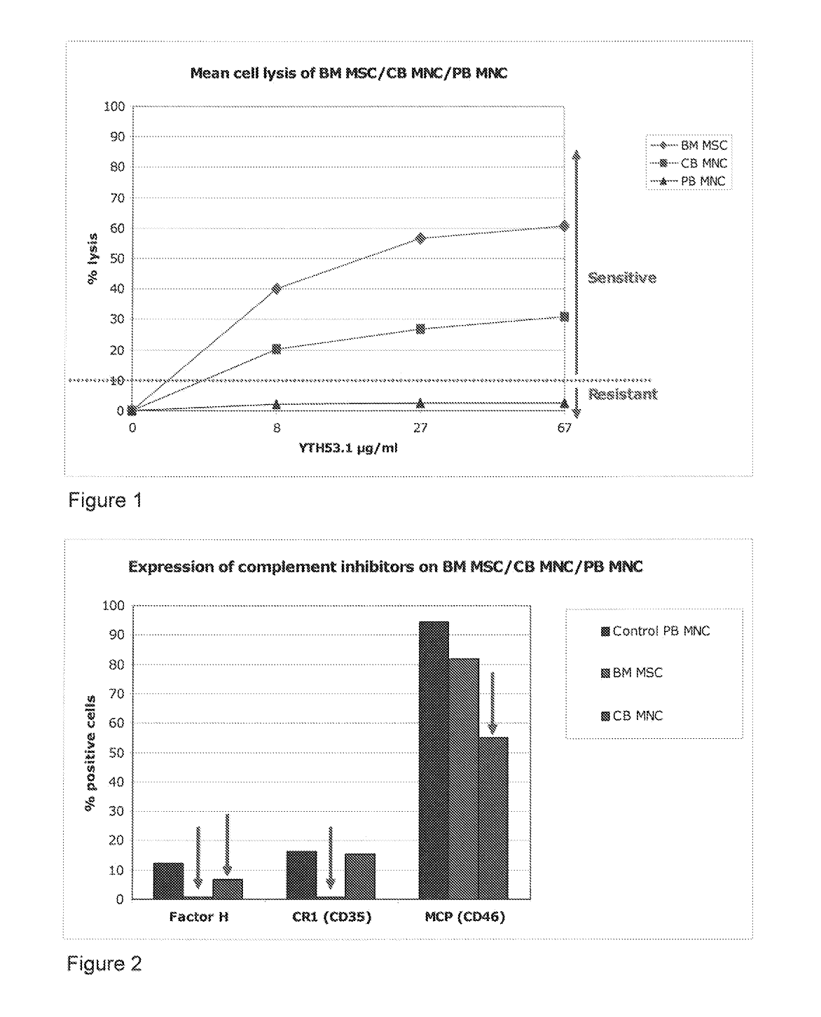

Bone marrow-derived mesenchymal stem cells and cord blood-derived mononuclear cells (including the CD34-positive hematopoietic stem cells) were sensitive to complement-mediated destruction with average lysis percentage above 50% and 25%, respectively. Peripheral blood-derived mononuclear cells that served as the control cell population were resistant to complement-mediated lysis with average lysis percentage of 2%. The results are presented in FIG. 1.

### Example 2

### Materials and Methods

Flow cytometric analysis: Cells were prepared as in Example 1. In flow cytometric analysis, cells were washed twice and suspended in PBS supplemented with 1% BSA. For each staining, 5×10⁵ cells were incubated at +22° C. for 20 minutes with 5 μg/ml of the appropriate primary monoclonal anti-body against complement inhibitors factor H (FH), complement receptor 1 (CR1) and membrane cofactor protein (MCP). After washing the cells three times, they were incubated for a further 30 minutes on ice with ALEXA⁴⁸⁸-conjugated goat anti-mouse F(ab′)₂. The cells were then washed again three times, fixed with 1% paraformaldehyde and analyzed on a Becton Dickinson FACScan 440 flow cytometer. Data were analyzed using the ProCOUNT™ software or Windows Multiple Document Interface for Flow Cytometry (WinMDI version 2.8).

### Results

The level of complement inhibitor factor H (FH) was markedly decreased on bone marrow-derived mesenchymal stem cells and on cord blood-derived mononuclear cells (including the CD34-positive hematopoietic stem cells). The expression of complement inhibitor complement receptor 1 (CR1) was extremely low on bone marrow-derived mesenchymal stem cells. The level of complement inhibitor membrane cofactor protein (MCP) was lower in cord blood-derived mononuclear cells when compared to peripheral blood-derived mononuclear cells that served as the control cell population. The results are presented in FIG. 2.

### Example 3

### Materials and Methods

Cells: Ficoll-Hypaque density gradient was used to isolate mononuclear cells from cord blood. Cord blood-derived CD34-positive cells were sorted from the mononuclear cell fraction with anti-CD34 microbeads by magnetic affinity cell sorting, and CD34-negative cells representing mature leukocytes were collected for control purposes.

Flow cytometric analysis: In flow cytometric analysis, cells were washed and suspended in PBS supplemented with 1% BSA. For each staining, 10⁵ cells were incubated for 15 minutes at RT with 5 μg/ml of the appropriate primary monoclonal antibody against complement inhibitors membrane cofactor protein (MCP, CD46), decay accelerating factor (DAF, CD55), and factor H (FH). The anti-FH antibody was directly conjugated with ALEXA⁴⁸⁸ fluorochrome. The anti-MCP and anti-DAF antibodies were biotinylated and they were used together with ALEXA⁴⁸⁸-avidin secondary antibody in a further incubation for 15 minutes at RT. The cells were then washed and analyzed on a Becton Dickinson FACScan flow cytometer. Data were analyzed using the CellQuest-Pro™ software.

### Results

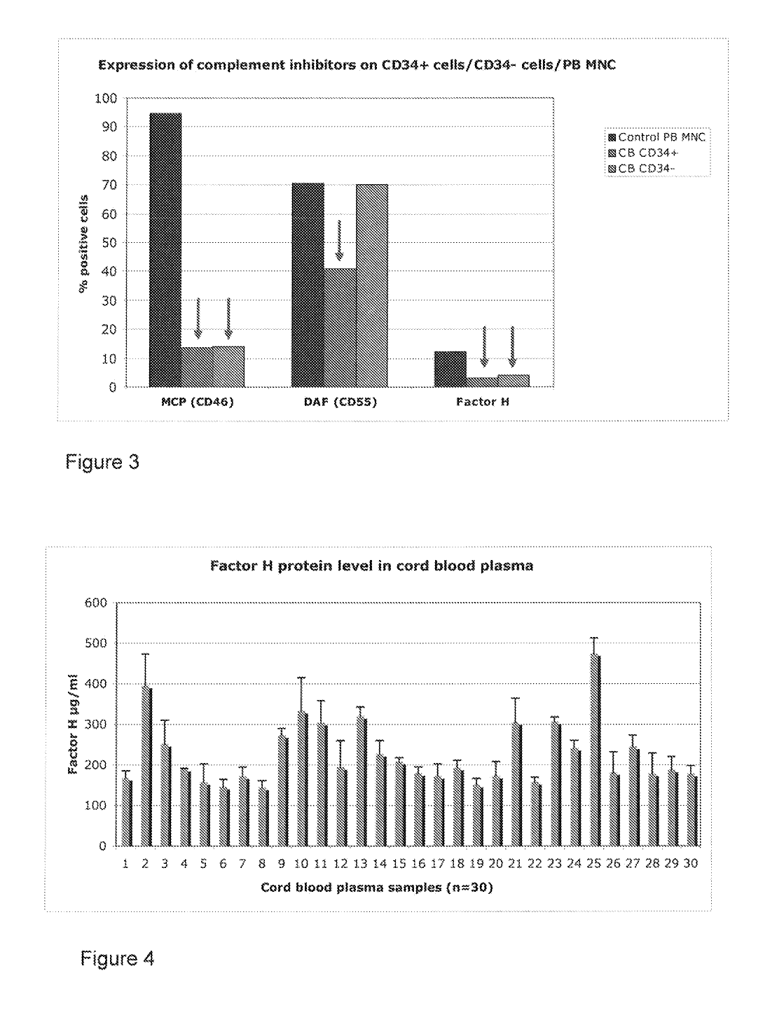

In cord blood-derived CD34-positive and CD34-negative cells, the levels of complement inhibitors membrane cofactor protein (MCP) and factor H (FH) were significantly decreased. In addition, the expression of complement inhibitor decay accelerating factor (DAF) was markedly lower in cord blood-derived CD34-positive cells when compared to peripheral blood-derived mononuclear cells or cord blood-derived CD34-negative cells. The results are presented in FIG. 3.

### Example 4

### Materials and Methods

Lysis assay: Cells were prepared as in Example 1. Labeling of cells was performed as described in Example 1. The effect of factor H on complement-mediated lysis of cells was studied by treating the cells with the complement-activating and CD59-neutralizing antibody (YTH53.1) alone, or in the presence of factor H (125-500 μg/ml). After centrifugation at 525×g for 5 minutes, 50% of the supernatant was carefully removed and counted in a gamma counter. Cell lysis was determined as percentage of specific release of ⁵¹Cr.

### Results

Complement-mediated lysis of bone marrow-derived mesenchymal stem cells was diminished by addition of complement inhibitor factor H. The results are presented in Table 1.

TABLE 1
Lysis sensitivity of bone marrow-derived mesenchymal stem cells
without/with factor H

| | % Lysis without | % Lysis with | Change in lysis |
|---|---|---|---|
| Factor H (μg/ml) | factor H | factor H | sensitivity |
| 125 | 84% | 80% | −5% |
| 250 | 70% | 52% | −26% |
| 500 | 70% | 60% | −14% |

### Example 5

### Materials and Methods

Lysis assay: Cells were prepared as in example 3. Labeling of cells was performed as described in example 1. The effect of factor H on complement-mediated lysis of cells was studied by treating the cells with the complement-activating and CD59-neutralizing antibody (YTH53.1) alone, or in the presence of factor H (500 μg/ml). After centrifugation at 525×g for 5 minutes, 50% of the supernatant was carefully removed and counted in a gamma counter. Cell lysis was determined as percentage of specific release of ⁵¹Cr.

### Results

Complement-mediated lysis of cord blood-derived hematopoietic stem cells, the CD34-positive cells, was significantly reduced by addition of complement inhibitor factor H. Further, factor H protected the CD34-negative cells from destruction as well. The results are presented in Table 2.

TABLE 2
Lysis sensitivity of cord blood-derived CD34+ and CD34− cells without/
with factor H

| Cord | | % Lysis without | % Lysis with | Change in ly- |
|---|---|---|---|---|
| blood unit | Sample | factor H | factor H | sis sensitivity |
| 1 | CD34+ | 12% | 0% | −100% |
| 1 | CD34− | 31% | 8% | −74% |
| 2 | CD34+ | 17% | 6% | −65% |
| 2 | CD34− | 64% | 23% | −64% |

### Example 6

### Materials and Methods

ELISA assay: To determine the amounts of factor H in the cord blood and peripheral blood, an ELISA assay was used. Microtiter plates (Nunc Polysorp, Denmark) were coated with a polyclonal goat-anti-human factor H antibody diluted 1:1,000 in carbonate buffer (15 mM Na₂CO₃, 35 mM NaHCO₃, pH 9.6). After an overnight incubation at +4° C., the wells were washed with 0.05% Tween/PBS and nonspecific binding sites were blocked by incubation with 1% BSA/PBS at room temperature for 1 h. The plates were then washed and the samples were applied diluted in 1% BSA/PBS. Purified factor H (Cal-biochem) in dilutions ranging between 3 and 3000 ng/ml was used as a standard curve. After a 2 h incubation at +37° C., the plates were washed and the monoclonal anti-factor H antibody 196X in 1% BSA/PBS (3 μg/ml) was added and incubated for 2 h at room temperature. 196X binds to the SCR1 domain of both factor H and the alternatively spliced protein FHL-1. After washing, the HRP-conjugated rabbit-anti-mouse IgG (Jackson), diluted 1:2000 in 0.05% Tween/PBS, was added and incubated at room temperature for 1 h. The plates were then washed and the substrate (OPD) was added. The color reaction was stopped with 0.5 M H2SO4 and the absorbance was measured at 492 nm.

### Results

An ELISA assay, employing the monoclonal antibody 196X against factor H and FHL-1, was used to determine the level of factor H and FHL-1 in cord blood and peripheral blood. The combined mean plasma level of factor H/FHL-1 in cord blood was 227±80 μg/ml (mean±SD; n=30), whereas it was 540±157 μg/ml (mean±SD; n=33) in normal human plasma. The results show that the level of the potent complement inhibitor factor H in cord blood plasma is only approximately 42% of its level in normal human plasma. This correlates with the findings in example 2 (the expression of factor H protein on cord blood mononuclear cells is 7.6%, whereas it is 12.3% on peripheral blood mononuclear cells).

There is variation in cord blood plasma factor H level between different cord blood units. Thus, some cord blood-derived stem cells may be more prone to complement-mediated lysis than others. This complement sensitivity, based on the factor H level in a certain cord blood unit, could be measured prior to cord blood transplantation. The results are presented in FIG. 4.

### Example 7

### Materials and Methods

Cells: Cord blood was collected in a multiple bag system containing 17 ml of citrate phosphate dextrose buffer (Cord Blood Collection System; Eltest, Bonn, Germany). Prior to the isolation of mononuclear cells, the anti-coagulated cord blood was diluted 1:2 with 2 mM EDTA-PBS. Mononuclear cells were isolated using Ficoll-Hypaque (Amersham Biosciences, Piscaway, N.J., USA) gradient centrifugation. 1×10⁶/cm² mononuclear cells were plated on fibronectin (Sigma) coated tissue culture plates (Nunc) in proliferation medium consisting of minimum essential medium a (aMEM) with Glutamax (Gibco, Grand Island, N.Y., USA) and 10% fetal calf serum (FCS) (Gibco) supplemented with 10 ng/ml epidermal growth factor (EGF, Sigma), 10 ng/ml recombinant human platelet-derived growth factor (rhPDGF-BB; R&D Systems, Minneapolis, Minn., USA), 50 nM Dexamethasone (Sigma), 100 U/ml penicillin+100 mg/ml streptomycin (Invitrogen). The initial cord blood-derived mesenchymal cell line establishment was performed in a humidified incubator with hypoxic conditions (5% CO₂, 3% O₂ and 37° C.). Cells were allowed to adhere overnight and non-adherent cells were washed out with medium changes. Proliferation media was renewed twice a week. Established CB MNC lines (391P, 392T, 454T) were passaged when almost confluent and replated at 1000-3000 cells/cm² in proliferation media in normoxic conditions (5% CO₂, 20% O₂ and 37° C.).

Lysis assay: Labeling of cells was performed by mixing 1-2×10⁶ cells and 50 μCi of ⁵¹Cr in 1 ml RPMI for 2 h at 37° C. The cells were then washed three times with RPMI, incubated for a further 30 minutes to remove loosely bound ⁵¹Cr and washed again three times with RPMI. Duplicate aliquots of ⁵¹Cr-labeled cells (10⁵ cells/50 μl) were treated with monoclonal anti-body against CD59 (YTH53.1) for 20 minutes at 22° C. and with normal human serum (NHS) for 30 minutes at 37° C. in a total volume of 200 μl. NHS was diluted 1:4 and YTH53.1 was used in concentrations 0.1-30 μg/ml. After centrifugation at 525×g for 5 minutes, 50% of the supernatant was carefully removed and counted in a gamma counter. Cell lysis was determined as percentage of specific release of ⁵¹Cr.

### Results

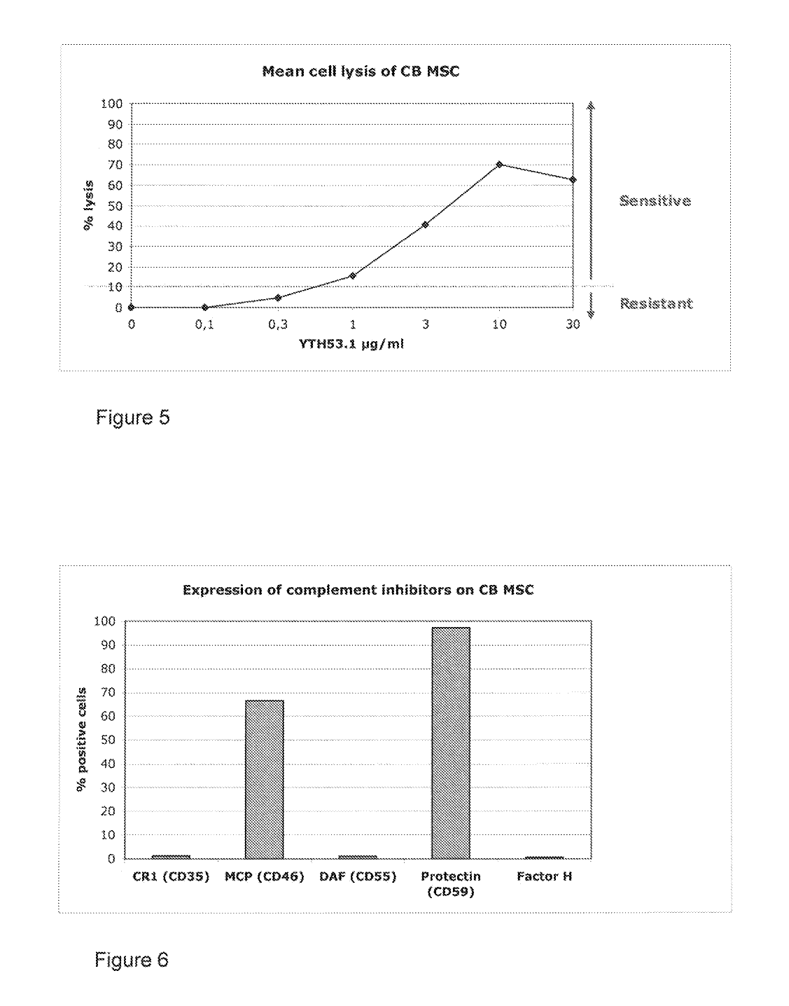

Cord blood-derived mesenchymal stem cells (391P) were sensitive to complement-mediated destruction with mean lysis percentage of 70%. The results are presented in FIG. 5.

### Example 8

### Materials and Methods

Lysis assay: Cord blood-derived mesenchymal cells 391P were prepared as in example 7. Labeling of cells was performed as described in example 7. The effect of factor H on complement-mediated lysis of cells was studied by treating the cells with the complement-activating and CD59-neutralizing antibody (YTH53.1) alone, or in the presence of factor H (10-100 μg/ml). After centrifugation at 525×g for 5 minutes, 50% of the supernatant was carefully removed and counted in a gamma counter. Cell lysis was determined as percentage of specific release of ⁵¹Cr.

### Results

Complement-mediated lysis of cord blood-derived mesenchymal stem cells (391P) was moderately diminished by low concentrations of complement inhibitor factor H. The results are presented in Table 3.

TABLE 3
Lysis sensitivity of cord blood-derived mesenchymal stem cells without/
with factor H

| Factor H (μg/ml) | % Lysis | Change in lysis sensitivity |
|---|---|---|
| 0 | 84.5% | — |
| 10 | 86.0% | — |
| 30 | 78.5% | −7.1% |
| 100 | 76.5% | −9.5% |

### Example 9

### Materials and Methods

Flow cytometric analysis: Cells were prepared as in Example 7. In flow cytometric analysis, cells were washed once and suspended in PBS supplemented with 1% BSA. For each staining, 5×10⁵ cells were incubated at +22° C. for 20 minutes with approximately 5 μg/ml of the appropriate ALEXA⁴⁸⁸- or FITC-conjugated antibodies against complement receptor 1 (CR1, CD35), membrane cofactor protein (MCP, CD46), decay accelerating factor (DAF, CD55), Protectin (CD59) and factor H (FH). The cells were then washed with PBS supplemented with 1% BSA and analyzed on a Becton Dickinson FAC-Scan 440 flow cytometer. Data were analyzed using the ProCOUNT™ software.

### Results

In cord blood-derived mesenchymal stem cells (391P), the levels of complement inhibitors complement receptor 1 (CR1, CD35), decay accelerating factor (DAF, CD55) and factor H (FH) were very low, when compared to the expression of membrane cofactor protein (MCP, CD46) and Protectin (CD59). The results are presented in FIG. 6.

1-15. (canceled)

16. A method for protecting stem cells in a clinical graft against destruction induced by complement system, comprising adding to the graft at least one factor capable of inhibiting the complement.

17. The method according to claim 16, wherein the complement system is activated by a nonhuman Neu5Gc structure on a stem cell surface.

18. The method according to claim 16, wherein the at least one factor capable of inhibiting the complement is selected from the group consisting of factor H, CR1, MCP, and DAF.

19. The method according to claim 16, wherein the stem cells comprise mesenchymal stem cells and/or hematopoietic stem cells.

20. A composition or a mixture, comprising stem cells and at least one factor capable of inhibiting complement.

21. The composition according to claim 20, wherein the at least one factor capable of inhibiting complement is selected from the group consisting of factor H, CR1, MCP, and DAF.

22. The composition according to claim 20, wherein the stem cells comprise mesenchymal stem cells and/or hematopoietic stem cells.

23. A method for protecting a cord blood derived cell against destruction induced by complement system, comprising adding to the cord blood derived cell at least one factor capable of inhibiting complement.

24. The method according to claim 23, wherein the complement system is activated by a nonhuman Neu5Gc structure on a cord blood derived cell surface.

25. A method of adjusting the amount of fortification of a complement inhibitor, comprising: first measuring a concentration of the complement inhibitor in a clinical graft; and adding a missing amount of the complement inhibitor thereto.

26. A method of adjusting the amount of fortification of a complement inhibitor, comprising: first measuring a concentration of the complement inhibitor in a clinical graft; and then administering a missing amount of the complement.
